(19) United States
(12) Patent Application Publication
(10) Pub. No.: US 2026/0094725 A1
(43) Pub. Date: Apr. 2, 2026

(54) COMPUTER SYSTEM AND METHOD FOR SIMULATION OF MEDICAL SCANS

(71) Applicant: MediSim Technologies, LLC, Orlando, FL (US)

(72) Inventor: Matthew Hayes, Cary, NC (US)

(73) Assignee: MediSim Technologies, LLC, Orlando, FL (US)

(21) Appl. No.: 19/345,824

(22) Filed: Sep. 30, 2025

Related U.S. Application Data
(60) Provisional application No. 63/701,368, filed on Sep. 30, 2024.

(51) Int. Cl. G16H 50/50; G06N 3/08; G06N 20/00; G09B 23/28; G16H 30/20; G16H 30/40; G16H 50/20; G16H 50/70

(52) U.S. Cl. CPC G16H 50/50; G06N 3/08; G06N 20/00; G09B 23/285; G09B 23/286; G16H 30/20; G16H 30/40; G16H 50/20; G16H 50/70

(57) ABSTRACT

A computer system and method for simulating one or more anatomical features of a simulated patient for application with a simulated medical scanning procedure. A medical scanning procedure by a medical scanning device is digitally simulated on a user selected simulated patient, wherein the user defines one or more anatomical features associated with the user selected patient. A computer display is generated enabling user selection of a certain simulated patient, including user selection of one or more anatomical features associated with the user selected simulated patient, for medical scanning simulation with a simulated medical scanning device. Simulation further includes digital simulation of injection of dye material into the simulated patient for application with a simulated medical scan requiring use of dye material.

### CROSS REFERENCE TO RELATED APPLICATIONS

This application claims priority to U.S. patent application Ser. No. 63/701,368 filed Sep. 30, 2024, which is incorporated herein by reference in its entirety.

### BACKGROUND

### Field

Computer medical simulation system and method, and more particularly, application of a simulated patient with a simulated medical scanning procedure.

### Description of Related Art

Simulation-based training is a well-recognized component in maintaining and improving skills. Consequently, simulation-based training is critically important for a number of professionals, such as airline pilots, fighter pilots, nurses and medical surgeons, among others. Such skills require hand-eye coordination, spatial awareness, and integration of multi-sensory input, such as tactile and visual. People in these professions have been shown to increase their skills significantly after undergoing simulation training.

A number of medical simulation products for training purposes are on the market. They include manikins for CPR training, obstetrics manikins, and manikins where chest tube insertion can be practiced, among others. There are manikins with an arterial pulse for assessment of circulatory problems or with varying pupil size for practicing endotracheal intubation. In addition, there are medical training systems for laparoscopic surgery practice, for surgical planning (based on three-dimensional imaging of the existing condition), and for practicing the acquisition of biopsy samples, to name just a few applications. Radiology imaging is the only interactive, real time imaging modality. For instance, much greater skill and experience is required for a sonographer to acquire and store ultrasound images for later analysis than for performing CT or MRI scanning. Effective radiology scanning and diagnosis based on ultrasound imaging requires anatomical understanding, knowledge of the appearance of pathologies and trauma, proper image interpretation relative to transducer position and orientation on the patient's body, the effect of compression on the patient's body by a transducer, and the context of the patient's symptoms.

Such skills are today primarily obtained through hands-on training in medical school, at radiology training programs, and at short courses. These training sessions are an expensive proposition because a number of live, healthy models, ultrasound imaging systems, and qualified trainers are needed, which detract from their normal diagnostic and revenue-generating activities. There are also not enough teachers to meet the demand because qualified radiology technicians and physicians are required to earn Continuing Medical Examination (“CME”) credits annually.

Various phantoms (e.g., manikins, etc.) have been developed and are widely used for medical training purposes, such as prostate phantoms, breast phantoms, fetal phantoms, phantoms for practicing placing IV lines, etc. There are major limitations to the use of these phantoms for radiology training purposes. First, they need to be used together with an available radiology scanner. Thus, such simulation training can only occur at the hospital and only when the radiology scanner is not otherwise used for patent examination. Second, with a few exceptions, there are no phantoms for training to recognize trauma, and actual pathology situations relative to the many unique anatomical and physiology features of an actual live patient. And most importantly, there currently exists critical limitations with actual current medical training regarding medical radiology scanning procedures, especially in view of the fact that radiology healthcare professionals typically cannot afford to make mistakes on a live patient, especially in regard to medical training and/or experimentation purposes. For instance, this is because, due to the adverse effects that would be caused to a patient in view of ionizing radiation and/or RF exposure, and medication/contrast exposure, that practicing on an actual would entail.

Hence, exiting training procedures are static procedures (and/or have specialized parts), and thus fall short of simulating a dynamic, interactive human. Given the ubiquitous use of radiology for medical diagnosis, and the large number of potential users, there is a large need for cost-effective radiology training. Training needs comes in several forms, including: (i) training active users in using new radiology scanners; (ii) training active users in new diagnostic procedures; (iii) training active users for re-certification, to maintain skills and earn continuing medical education credit on an annual basis; and (iv) training new users, such as primary care physicians, emergency medicine personnel, paramedics and EMTs.

What is needed is a better system and method of use that train medical scanning operators on a wide range of diagnostic subjects in a cost-effective, realistic, and consistent way.

### SUMMARY

The purpose and advantages of the below described illustrated embodiments will be set forth in and apparent from the description that follows. Additional advantages of the illustrated embodiments will be realized and attained by the devices, systems and methods particularly pointed out in the written description and claims hereof, as well as from the appended drawings.

To achieve these and other advantages and in accordance with the purpose of the illustrated embodiments, in one aspect, described is a computer medical simulation system and method for generating a simulated (e.g., digitally rendered) patient for application with a simulated medical scanning procedure. The illustrated embodiments generally provide a computer system and method operative and configured to digitally replicate a clinical environment for a medical training simulation. The embodiments described herein overcome and exceed the limitations of actual current medical training regarding medical radiology scanning procedures, especially in view of the fact that radiology healthcare professionals typically cannot make mistakes on a live patient, especially in regard to medical training and/or experimentation purposes. For instance, this is because, due to the adverse effects that would be caused to a patient in view of ionizing radiation and/or RF exposure, and medication/contrast exposure, that practicing on an actual would entail.

It is to be appreciated and understood, the illustrated embodiments additionally enable simulation of various other medical modalities that are based on physiology of the patient, rather than solely on the medical diagnostic equipment. The illustrated embodiments generate a digital simulated patient, that preferably combines 3D modeling of external and internal patient systems with independent patient physiology profiles. Thus, for instance, a digital simulated patient is simulated to have physiologic combinations of heart rhythm, structural variants, range of motion, cardiac output, peristalsis rate, and kidney function, while having psychologic conditions, such as for example, claustrophobia and nervousness.

Certain embodiments include a digital repository/database of simulate anatomical patient data utilized by the system and method to change the appearance of a simulated patient to mimic the appearance of other radiologic modalities, such as (and not limited to) PET, Nuclear medicine, and Bone Density medical scans. Additionally, certain embodiments include a digital injector configured and operative to be combined with simulated patient cardiovascular physiology so as to be utilized to determine an amount of time required for simulated imaging contrast (dye) to circulate through a simulated patient's cardiovascular system.

Accordingly, it is to be understood and appreciated that the certain illustrated embodiments provide two-deep simulations, providing a simulated scanning interface, and a simulated human patient.

### BRIEF DESCRIPTION OF THE DRAWINGS

So that those skilled in the art to which the subject disclosure appertains will readily understand how to make and use the devices and methods of the subject disclosure without undue experimentation, preferred illustrated embodiments thereof will be described in detail herein below with reference to certain figures, wherein:

FIG. 1 illustrates an example communication network utilized with one or more of the illustrated embodiments;

FIG. 2 illustrates an example network device/node utilized with one or more of the illustrated embodiments;

FIG. 3 illustrates a diagram depicting an Artificial Intelligence (AI) device utilized with one or more of the illustrated embodiments;

FIG. 4 illustrates a diagram depicting an AI server utilized with one or more of the illustrated embodiments;

FIGS. 5A and 5B illustrate digitally rendered dye injector systems in accordance with one or more of the illustrated embodiments;

FIG. 6 illustrates digitally rendered dye injector systems for use with a simulated examination procedure in accordance with one or more of the illustrated embodiments;

FIG. 7 illustrates stored data in a data repository representative of various patient outer profile anatomical shells in accordance with the illustrated embodiments;

FIGS. 8A-8C illustrates various rendered GUIs digitally replicating medical scanning procedures with a digitally rendered patient in accordance with the illustrated embodiments;

FIG. 9 illustrates various digitally rendered patient anatomical components in accordance with the illustrated embodiments;

FIG. 10 illustrates a simulated medical scanning procedure performed upon a simulated patient illustrating slicing scanning results of a 3D simulated patient model in accordance with the illustrated embodiments;

FIGS. 11A and 11B, and 12A and 12B illustrate simulated blood flow dynamics in simulated patient medical scans in accordance with the illustrated embodiments;

FIG. 13 illustrates a generated GUI of a medical simulated scanning system depicting user control of dye contrast in a simulated patient in accordance with the illustrated embodiments; and

FIG. 14 illustrates a generated GUI of a medical simulated scanning system depicting 3D modelling of simulated patient in accordance with the illustrated embodiments.

### DESCRIPTION OF CERTAIN EMBODIMENTS

The illustrated embodiments are now described more fully with reference to the accompanying drawings wherein like reference numerals identify similar structural/functional features. The illustrated embodiments are not limited in any way to what is illustrated as the illustrated embodiments described below are merely exemplary, which can be embodied in various forms, as appreciated by one skilled in the art. Therefore, it is to be understood that any structural and functional details disclosed herein are not to be interpreted as limiting, but merely as a basis for the claims and as a representation for teaching one skilled in the art to variously employ the discussed embodiments. Furthermore, the terms and phrases used herein are not intended to be limiting but rather to provide an understandable description of the illustrated embodiments.

Unless defined otherwise, all technical and scientific terms used herein have the same meaning as commonly understood by one of ordinary skill in the art to which this illustrated embodiment belongs. Although any methods and materials similar or equivalent to those described herein can also be used in the practice or testing of the illustrated embodiments, exemplary methods and materials are now described.

It must be noted that as used herein and in the appended claims, the singular forms “a”, “an,” and “the” include plural referents unless the context clearly dictates otherwise. Thus, for example, reference to “a stimulus” includes a plurality of such stimuli and reference to “the signal” includes reference to one or more signals and equivalents thereof known to those skilled in the art, and so forth.

It is to be appreciated the illustrated embodiments discussed below are preferably a software algorithm, program or code residing on computer useable medium having control logic for enabling execution on a machine having a computer processor. The machine typically includes memory storage configured to provide output from execution of the computer algorithm or program.

As used herein, the term “software” is meant to be synonymous with any code or program that can be in a processor of a host computer, regardless of whether the implementation is in hardware, firmware or as a software computer product available on a disc, a memory storage device, or for download from a remote machine. The embodiments described herein include such software to implement the equations, relationships and algorithms described above. One skilled in the art will appreciate further features and advantages of the illustrated embodiments based on the above-described embodiments. Accordingly, the illustrated embodiments are not to be limited by what has been particularly shown and described, except as indicated by the appended claims.

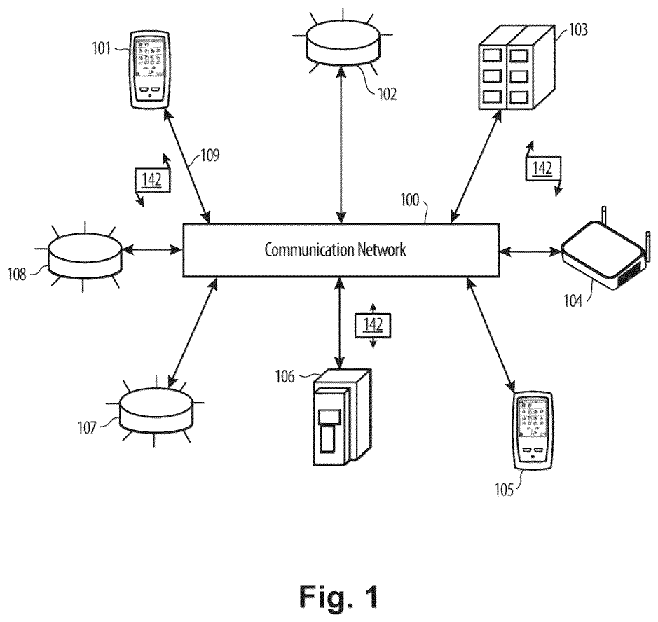

Turning now descriptively to the drawings, in which similar reference characters denote similar elements throughout the several views, FIG. 1 depicts an exemplary communications network 100 in which below illustrated embodiments may be implemented. It is to be understood a communication network 100 is a geographically distributed collection of nodes interconnected by communication links and segments for transporting data between end nodes, such as personal computers, work stations, smart phone devices, tablets, televisions, sensors and or other devices such as automobiles, etc. Many types of networks are available, with the types ranging from local area networks (LANs) to wide area networks (WANs). LANs typically connect the nodes over dedicated private communications links located in the same general physical location, such as a building or campus. WANs, on the other hand, typically connect geographically dispersed nodes over long-distance communications links, such as common carrier telephone lines, optical lightpaths, synchronous optical networks (SONET), synchronous digital hierarchy (SDH) links, or Powerline Communications (PLC), and others.

FIG. 1 is a schematic block diagram of an examplary communication network 100 illustratively comprising nodes/devices 101-108 (e.g., sensors 102, computing devices 103 (e.g., a medical computer simulation device), smart phone devices 105, web servers / computer systems 106, computer systems 107, switches 108, databases, and the like) interconnected by various methods of communication. For instance, the links 109 may be wired links or may comprise a wireless communication medium, where certain nodes are in communication with other nodes, e.g., based on distance, signal strength, current operational status, location, etc. Moreover, each of the devices can communicate data packets (or frames) 142 with other devices using predefined network communication protocols as will be appreciated by those skilled in the art, such as various wired protocols and wireless protocols etc., where appropriate. In this context, a protocol consists of a set of rules defining how the nodes interact with each other. Those skilled in the art will understand that any number of nodes, devices, links, etc. may be used in the computer network, and that the view shown herein is for simplicity. Also, while the embodiments are shown herein with reference to a general network cloud, the description herein is not so limited, and may be applied to networks that are hardwired.

As will be appreciated by one skilled in the art, aspects of the illustrated embodiments may be embodied as a system, method or computer program product. Accordingly, aspects of the illustrated embodiments may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the illustrated embodiments may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device. A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing. Computer program code for carrying out operations for aspects of the illustrated embodiments may be written in any combination of one or more programming languages, including an object oriented programming language such as Python, Golang, Ruby, ASP.NET, Java, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the illustrated embodiments are described below with reference to flowchart illustrations and/or block diagrams of methods, apparatus (systems) and computer program products according to the illustrated embodiments. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

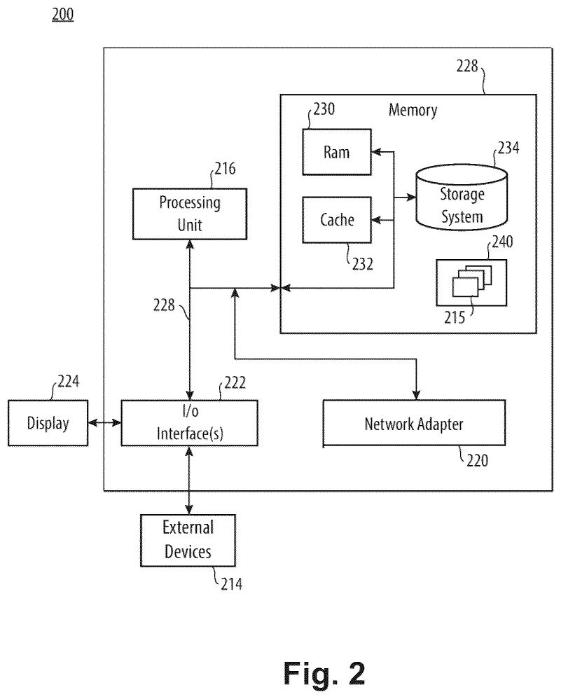

FIG. 2 is a schematic block diagram of an example network computing device 200 (e.g., a medical simulation device 103) that may be used (or components thereof) with one or more embodiments described herein, e.g., as one of the nodes shown in the network 100. As explained above, in different embodiments these various devices are configured to communicate with each other in any suitable way, such as, for example, via communication network 100.

Device 200 is intended to represent any type of computer system capable of carrying out the teachings of various illustrated embodiments. Device 200 is only one example of a suitable system and is not intended to suggest any limitation as to the scope of use or functionality of the illustrated embodiments described herein. Regardless, computing device 200 is capable of being implemented and/or performing any of the functionality set forth herein, including in a medical simulation device 103.

Computing device 200 is operational with numerous other general purpose or special purpose computing system environments or configurations. Examples of well-known computing systems, environments, and/or configurations that may be suitable for use with computing device 200 include, but are not limited to, cloud computing systems (including, but not limited to: Infrastructure as a Service (Iaas); Software as a Service (SaaS); Platform as a Service (PaaS); and Private cloud), personal computer systems, server computer systems, thin clients, thick clients, hand-held or laptop devices, multiprocessor systems, microprocessor-based systems, programmable consumer electronics, network PCs, minicomputer systems, and distributed data processing environments that include any of the above systems or devices, and the like. Computing device 200 may be described in the general context of computer system-executable instructions, such as program modules, being executed by a computer system. Generally, program modules may include routines, programs, objects, components, logic, data structures, and so on that perform particular tasks or implement particular abstract data types. Computing device 200 may be practiced in distributed data processing environments where tasks are performed by remote processing devices that are linked through a communications network. In a distributed data processing environment, program modules may be located in both local and remote computer system storage media including memory storage devices. In accordance with the illustrated embodiments, computing device 200 is configured and operative, relative to a medical simulation device 103, to generate a simulated patient for application with a simulated medical scanning procedure.

The components of device 200 may include, but are not limited to, one or more processors or processing units 216, a system memory 228, and a bus 218 that couples various system components including system memory 228 to processor 216. Bus 218 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus. Computing device 200 typically includes a variety of computer system readable media. Such media may be any available media that is accessible by device 200, and it includes both volatile and non-volatile media, removable and non-removable media.

System memory 228 can include computer system readable media in the form of volatile memory, such as random-access memory (RAM) 230 and/or cache memory 232. Computing device 200 may further include other removable/non-removable, volatile/non-volatile computer system storage media. By way of example only, storage system 234 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 218 by one or more data media interfaces. As will be further depicted and described below, memory 228 may include at least one program product having a set (e.g., at least one) of program modules that are configured to carry out the functions of embodiments of illustrated embodiments.

Program/utility 240, having a set (at least one) of program modules 215, such as underwriting module, may be stored in memory 228 by way of example, and not limitation, as well as an operating system, one or more application programs, other program modules, and program data. Each of the operating system, one or more application programs, other program modules, and program data or some combination thereof, may include an implementation of a networking environment. Program modules 215 generally carry out the functions and/or methodologies of the illustrated embodiments as described herein, including, but not limited to to generate a simulated patient for application with a simulated medical scanning procedure, preferably via a medical simulation device/system 103, as described further below.

Device 200 may also communicate with one or more external devices 214 such as a keyboard, a pointing device, a display 224, etc. ; one or more devices that enable a user to interact with computing device 200; and/or any devices (e.g., network card, modem, etc.) that enable computing device 200 to communicate with one or more other computing devices. Such communication can occur via Input/Output (I/O) interfaces 222. Still yet, device 200 can communicate with one or more networks such as a local area network (LAN), a general wide area network (WAN), and/or a public network (e.g., the Internet) via network adapter 220. As depicted, network adapter 220 communicates with the other components of computing device 200 via bus 218. It should be understood that although not shown, other hardware and/or software components could be used in conjunction with device 200. Examples, include, but are not limited to: big data technologies encompassing large and diverse datasets that are significant in volume, which are commonly used in machine learning, predictive modeling, and other advanced analytics to solve business problems and make informed decisions; non-relational databases (NoSQLs); Blob storage; relational databases (SQL); as well as microcode, device drivers, redundant processing units, external disk drive arrays, RAID systems, tape drives, and data archival storage systems, etc.

FIGS. 1 and 2 are intended to provide a brief, general description of an illustrative and/or suitable exemplary environment in which the below described illustrated embodiments may be implemented. FIGS. 1 and 2 are exemplary of a suitable environment and are not intended to suggest any limitation as to the structure, scope of use, or functionality of an illustrated embodiment. A particular environment should not be interpreted as having any dependency or requirement relating to any one or combination of components illustrated in an exemplary operating environment. For example, in certain instances, one or more elements of an environment may be deemed not necessary and omitted. In other instances, one or more other elements may be deemed necessary and added.

It is to be understood the embodiments described herein are preferably provided with self-learning/Artificial Intelligence (AI) to generate a simulated patient for application with a simulated medical scanning procedure by a medical simulation device/system 103, as described herein. Thus, preferably integrated into a medical simulation device/system, coupled to a plurality of external databases/data sources is an AI system (e.g., an Expert System) that implements machine learning and artificial intelligence algorithms to conduct one or more of the above-mentioned tasks for simulating medical scanning procedures using simulated patients, as described herein. For instance, the AI system may include two subsystems: a first sub-system that learns from historical data; and a second subsystem to identify and recommend one or more parameters or approaches based on the learning. It should be appreciated that although the AI system may be described as two distinct subsystems, the AI system can also be implemented as a single system incorporating the functions and features described with respect to both subsystems.

In accordance with the illustrated embodiments described herein, artificial intelligence refers to the field of studying artificial intelligence or methodology for making artificial intelligence, and machine learning refers to the field of defining various issues dealt with in the field of artificial intelligence and studying methodology for solving the various issues. Machine learning is defined as an algorithm that enhances the performance of a certain task through a steady experience with the certain task.

Also, in accordance with the illustrated embodiments, an artificial neural network (ANN) is a model used in machine learning and may mean a whole model of problem-solving ability which is composed of artificial neurons (nodes) that form a network by synaptic connections. The artificial neural network can be defined by a connection pattern between neurons in different layers, a learning process for updating model parameters, and an activation function for generating an output value. The artificial neural network may include an input layer, an output layer, and optionally one or more hidden layers. Each layer includes one or more neurons, and the artificial neural network may include a synapse that links neurons to neurons. In the artificial neural network, each neuron may output the function value of the activation function for input signals, weights, and deflections input through the synapse.

Model parameters refer to parameters determined through learning and include a weight value of synaptic connection and deflection of neurons. A hyperparameter means a parameter to be set in the machine learning algorithm before learning, and includes a learning rate, a repetition number, a mini batch size, and an initialization function. The purpose of the learning of the artificial neural network may be to determine the model parameters that minimize a loss function. The loss function may be used as an index to determine optimal model parameters in the learning process of the artificial neural network. Machine learning may be classified into supervised learning, unsupervised learning, and reinforcement learning according to a learning method. The supervised learning may refer to a method of learning an artificial neural network in a state in which a label for learning data is given, and the label may mean the correct answer (or result value) that the artificial neural network must infer when the learning data is input to the artificial neural network. The unsupervised learning may refer to a method of learning an artificial neural network in a state in which a label for learning data is not given. The reinforcement learning may refer to a learning method in which an agent defined in a certain environment learns to select a behavior or a behavior sequence that maximizes cumulative compensation in each state.

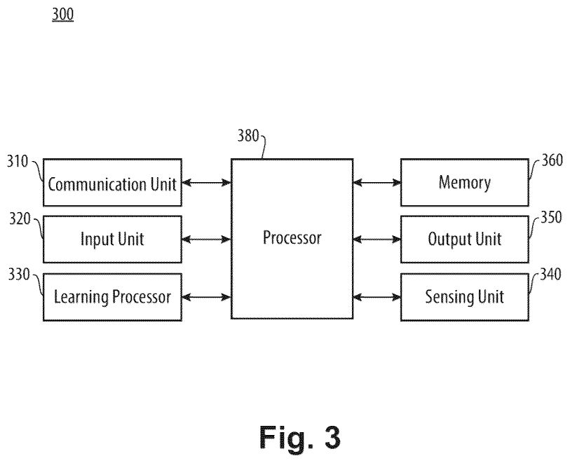

Machine learning, which is implemented as a deep neural network (DNN) including a plurality of hidden layers among artificial neural networks, is also referred to as deep learning, and the deep learning is part of machine learning. FIG. 3 illustrates an AI device 300 according to an illustrated embodiment. In accordance with the illustrated embodiments, the AI device 300 is preferably integrated into in verification computer system 103.

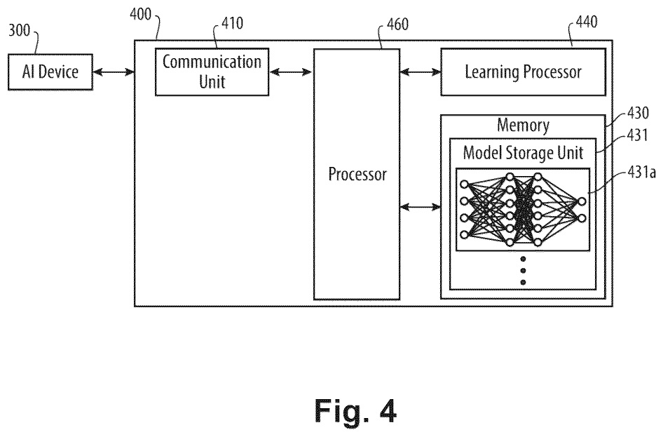

Referring now FIG. 3, in conjunction with FIGS. 1 and 2, the AI device 300 is operatively coupled to, or integrated with computing device 200, in accordance with the illustrated embodiments described herein. AI device 300 preferably includes a communication unit 310, an input unit 320, a learning processor 330, a sensing unit 340, an output unit 350, a memory 370, and a processor 380. The communication unit 310 may transmit and receive data to and from external devices such as other AI devices 300a to 300e and an AI server 400 (FIG. 4) by using wire/wireless communication technology. For example, the communication unit 310 may transmit and receive sensor information, a user input, a learning model, and a control signal to and from external devices.

The communication technology used by the communication unit 310 preferably includes GSM (Global System for Mobile communication), CDMA (Code Division Multi Access), LTE (Long Term Evolution), 5G, WLAN (Wireless LAN), Wi-Fi (Wireless-Fidelity), Bluetooth™, RFID (Radio Frequency Identification), Infrared Data Association (IrDA), ZigBee, NFC (Near Field Communication), and the like.

The input unit 320 may acquire various kinds of data, including, but not limited to medical scanning procedures/devices and/or patient anatomical data for providing simulation of one or more medical scanning procedures on one or more simulated patients. The input unit 320 may acquire a learning data for model learning and an input data to be used when an output is acquired by using learning model. The input unit 320 may acquire raw input data. In this case, the processor 380 or the learning processor 330 may extract an input feature by preprocessing the input data. The learning processor 330 may learn a model composed of an artificial neural network by using learning data. The learned artificial neural network may be referred to as a learning model. The learning model may be used to an infer result value for new input data rather than learning data, and the inferred value may be used as a basis for determination to perform a certain operation.

At this time, the learning processor 330 may perform AI processing together with the learning processor 330 of the AI server 400, and the learning processor 330 may include a memory integrated or implemented in the AI device 300. Alternatively, the learning processor 330 may be implemented by using the memory 370, an external memory directly connected to the AI device 300, or a memory held in an external device. The sensing unit 340 may acquire at least one of internal information about the AI device 300, ambient environment information about the AI device 300, and user information by using various sensors.

The output unit 350 preferably includes a display unit for outputting/displaying relevant information to a user in accordance with the illustrated embodiments described herein. The memory 370 preferably stores data that supports various functions of the AI device 300. For example, the memory 370 may store input data acquired by the input unit 320, learning data, a learning model, a learning history, and the like.

The processor 380 preferably determines at least one executable operation of the AI device 300 based on information determined or generated by using a data analysis algorithm or a machine learning algorithm. The processor 380 may control the components of the AI device 300 to execute the determined operation. To this end, the processor 380 may request, search, receive, or utilize data of the learning processor 330 or the memory 370. The processor 380 may control the components of the AI device 300 to execute the predicted operation or the operation determined to be desirable among the at least one executable operation. When the connection of an external device is required to perform a determined operation, the processor 380 may generate a control signal for controlling the external device and may transmit the generated control signal to the external device. The processor 380 may acquire intention information for the user input and may determine the user's requirements based on the acquired intention information. The processor 380 may acquire the intention information corresponding to the user input by using at least one of a speech to text (STT) engine for converting speech input into a text string or a natural language processing (NLP) engine for acquiring intention information of a natural language.

At least one of the STT engine or the NLP engine may be configured as an artificial neural network, at least part of which is learned according to the machine learning algorithm. At least one of the STT engine or the NLP engine may be learned by the learning processor 330, may be learned by the learning processor 340 of the AI server 400, or may be learned by their distributed processing. The processor 380 may collect history information including the operation contents of the AI device 300 or the user's feedback on the operation and may store the collected history information in the memory 370 or the learning processor 330 or transmit the collected history information to the external device such as the AI server 400. The collected history information may be used to update the learning model.

The processor 380 may control at least part of the components of AI device 300 so as to drive an application program stored in memory 370. Furthermore, the processor 380 may operate two or more of the components included in the AI device 300 in combination so as to drive the application program.

FIG. 4 illustrates an AI server 400 according to the illustrated embodiments. It is to be appreciated that the AI server 400 may refer to a device that learns an artificial neural network by using a machine learning algorithm or uses a learned artificial neural network. The AI server 400 may include a plurality of servers to perform distributed processing or may be defined as a 5G network. At this time, the AI server 400 may be included as a partial configuration of the AI device 300, and may perform at least part of the AI processing together. The AI server 400 may include a communication unit 410, a memory 430, a learning processor 440, a processor 460, and the like. The communication unit 410 can transmit and receive data to and from an external device such as the AI device 300. The memory 430 may include a model storage unit 431. The model storage unit 431 may store a learning or learned model (or an artificial neural network 431a) through the learning processor 440.

The learning processor 440 may learn the artificial neural network 431a by using the learning data. The learning model may be used in a state of being mounted on the AI server 400 of the artificial neural network or may be used in a state of being mounted on an external device such as the AI device 300. The learning model may be implemented in hardware, software, or a combination of hardware and software. If all or part of the learning models are implemented in software, one or more instructions that constitute the learning model may be stored in memory 430. The processor 460 may infer the result value for new input data by using the learning model and may generate a response or a control command based on the inferred result value.

It is to be understood and appreciated that FIGS. 1-4 are intended to provide a brief, general description of an illustrative and/or suitable exemplary environment in which the below described illustrated embodiments may be implemented. FIGS. 1-4 are exemplary of a suitable environment and are not intended to suggest any limitation as to the structure, scope of use, or functionality of an illustrated embodiment. A particular environment should not be interpreted as having any dependency or requirement relating to any one or combination of components illustrated in an exemplary operating environment. For example, in certain instances, one or more elements of an environment may be deemed not necessary and omitted. In other instances, one or more other elements may be deemed necessary and added.

It is to be understood and appreciated that the medical simulation device/system 103 as described herein, in certain embodiments, utilizes one or more artificial learning (AI) and/or machine learning techniques (FIGS. 3 and 4) for enabling the simulation of the one or more anatomical features of a simulated patient for application with a simulated medical scanning procedure, as described herein.

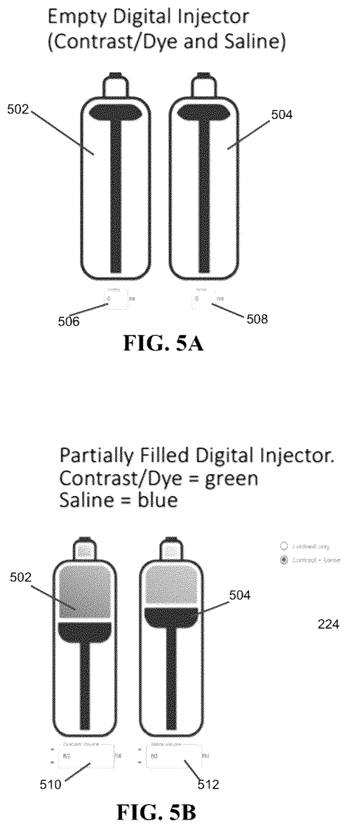

With the exemplary communication network 100 (FIG. 1), computing device 200 (FIG. 2), AI device 300 (FIG. 3) and AI server 400 (FIG. 4) being generally shown and discussed above, description of certain illustrated embodiments will now be provided with below reference to FIG. 5

With reference now to FIGS. 5-16 (and with continuing reference to FIGS. 1-4), generally described and shown is a computer-implemented system and method, via medical simulation system 103, for simulating one or more anatomical features of a simulated patient for application with a simulated medical scanning procedure, which in certain embodiments includes simulating one or more medical modalities associated with the simulated patient (which may be contingent upon a patient's physiology). The medical simulation system 103 is preferably operative and configured to generate simulated one or more anatomical features of a simulated patient, including three-dimensional (3D) modeling of the patient, wherein the 3D modeling includes modeling external and/or internal anatomical features of the simulated patient, and in certain embodiments further includes determination/generation of independent patient physiology profiles. In certain embodiments, the simulated patient includes one or more physiologic features, wherein the one or more physiologic features may include, for instance, one or more combinations of: heart rhythm, structural variants, range of motion, cardiac output, peristalsis rate, and/or kidney functions. Additionally, the one or more simulated physiologic features may further include psychological conditions, including claustrophobia and/or nervousness.

The medical simulation system 103 preferably includes a database repository (e.g., 228) for storing the one or more anatomical features of a simulated patient for subsequent user selection, which may be provided on a graphic user interface (GUI) provided on a computer display (e.g., 224) for user selection thereof. Preferably, the database repository is further configured and operative to change, via user selection, the appearance of the simulated patient to simulate/mimic user selected radiologic modalities. For instance, the simulated user selected radiologic modalities include (but are not to be understood to be limited to): positron emission tomography (PET) scans, nuclear medicine scans, and bone density scans.

In certain embodiments, the medical simulation system 103 is operative and configured to provide a computer-generated digital injector configured and operative to provide simulated injection of dye material into the simulated patient for application with a simulated medical scan requiring use of dye material. For instance, the simulated medical scanning procedure includes injection simulation of dye material into a patient to facilitate determination of the amount of time the imaging contrast, provided by the simulated dye, circulates through a simulated cardiovascular system of a simulated patient. It is to be understood and appreciated the simulated medical scan in certain embodiments requires use of simulated injected dye material, which may include for instance, a computed tomography (CT) scan, a radiology scan and/or a magnetic resonance imaging (MRI) scan. With regard to a simulated radiology scan, in certain embodiments the radiology scan is selected from the group consisting of one or more of the following scan types: MRI; CT; Ultrasound; X-ray; Mammography; PET; Fluoroscopy; Nuclear medicine; Bone scintigraphy Mammogram; Angiography Radiography; Diagnostic imaging; Computed axial tomography′ and Virtual colonoscopy. Thus, the simulated medical scanning procedure including injection simulation of dye material provides two computer simulations, one involving a GUI scanning interface, and another involving a simulated patient.

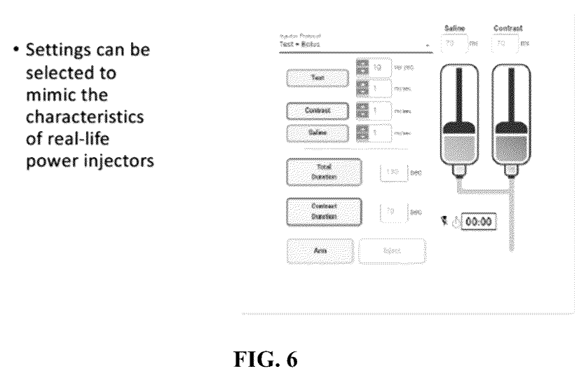

With specific reference now to FIGS. 5-14 (and with continuing reference to FIGS. 1-4), the medical simulation system 103 is configured and operative, as shown (and further described) in FIGS. 5 and 6, to generate a simulated digital injector (which is preferably replicated on a GUI of a computer display (e.g., 224)) providing one or more user interactive manipulable features for prescribing simulated injection of simulated dye 502 material into a simulated patient, including (and not limited to) prescribing an amount of saline and contrast fluid material to be injected in a simulated patient. In particular, display 224 of FIG. 5A depicts both the contrast/dye injector 502 and saline injector 504 as being empty (e.g., 506, 508), wherein the contrast/dye injector 502 and saline injector 504 are partially filled (e.g., 510, 512). FIG. 6 depicts the medical simulation system 103 generating a display 224 preferably having user controls (e.g., 514-524) for prescribing settings to be user selected for mimicking characteristics of actual power controlled saline 504 and dye injectors 502, including, but not limited to, a test injection volume and time 526, a contrast/dye time injection period 528, saline time injection period 530, a total duration period 532, and a contrast duration period 534, along with a simulated amount of lapse time 536 for simulated injection of contrast/dye 502 and saline 504.

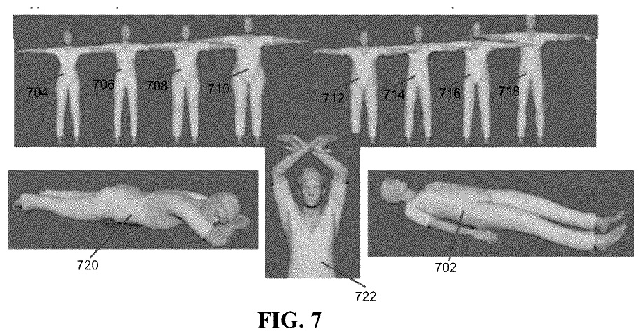

With reference now to FIG. 7, shown (and further described) is the above-mentioned database repository (e.g., 228) containing virtual outer shells of a plurality of simulated patients (e.g., 702-722) representative of different simulated patient: sexes; ages; body types and simulated patient bodies modeled in different positions. For instance, the aforesaid plurality of simulated patients, preferably generated by system 103, depicts various virtual outer shells for mimicking different sexes, ages, and body types, as well as being modelled to assume different positions.

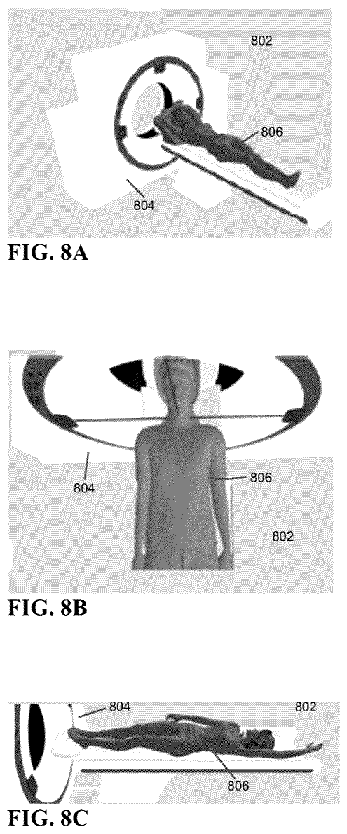

As shown (and further described) in FIGS. 8A-8C, the medical simulation system 103 is operative and configured to replicate (preferably on a GUI (e.g., 224)) a medical scanning environment 802, including a simulated medical scanning device 804. It is noted, stored in a database coupled to the medical simulation system 103 are a plurality of commercially available medical scanning devices from a third party vendor (e.g., 804) (e.g., GE, Hitachi, Philips, Siemens, United, etc.) available for user selection (e.g., via a GUI on a computer display (e.g., 224)) for use/application in a simulated medical scanning procedure, for use/application with a user selected/defined simulated patient (as described herein (e.g., 702). Thus, a user is enabled to select (preferably via user interaction on a computer display 224), MRI settings associated with a third party MRI vendor selected from a listing of certain third party vendors so as to generate on a GUI (e.g., computer display 224), a virtual MRI interface providing a same interface as associated with actual MRI scanner of the selected third party vendor.

As shown, the simulated medical scanning procedure is further operative and configured to graphically replicate application of a simulated patient 806 to the simulated medical scanning device 804, wherein the simulated patient 804 is positionable in a plurality of different body positions during a simulated medical scanning procedure. In particular, the medical system 103 is operative and configured to enable a user for prescribing simulated patients to be placed in, so as to interact with, digital (e.g., simulated) clinical environment.

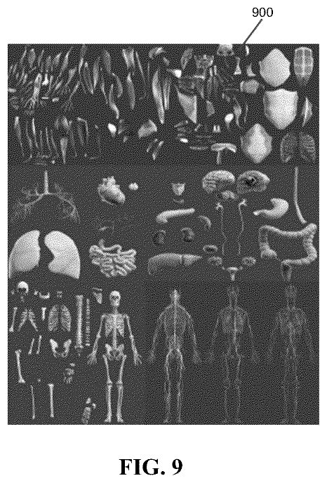

With reference to FIG. 9, shown (and further described) is the medical simulation device 103 being operative and configured to simulate one or more anatomical features of a simulated patient 900 on a computer display (e.g., 224), which for instance includes a plurality of different human tissue types and organs, which may be segmented to improve medical accuracy of certain simulated anatomical structures of the simulated patient 702. In accordance with the illustrated embodiments, the generated anatomical features of a simulated patient 900 are preferably rendered in 3D, wherein preferably each tissue type and organ are segmented and altered for improving medical accuracy of such anatomical structures. In certain embodiments, additional complex models are acquired to enhance accuracy and/or functionality.

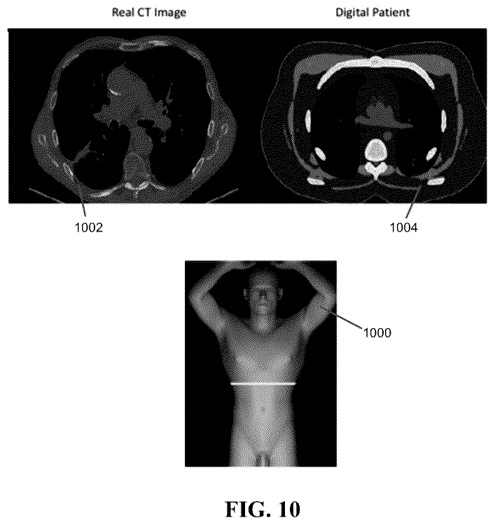

Turning to FIG. 10, the medical simulation device 103 is shown to be operative and configured to simulate, on a computer display 224, one or more anatomical features of a simulated patient which includes generation of a 3D model of a simulated patient 1000 that can be sliced in a multitude of directions in a 3D space (e.g., 10002 and 1004) for generating resulting images having an appearance to actual, non-simulated, medical images. The medical simulation device 103 is operative and configured, such that the 3D patient model (e.g., 1000) can be sliced in any direction in a 3D space such that resulting images appear with similar appearance to actual real-life medical images.

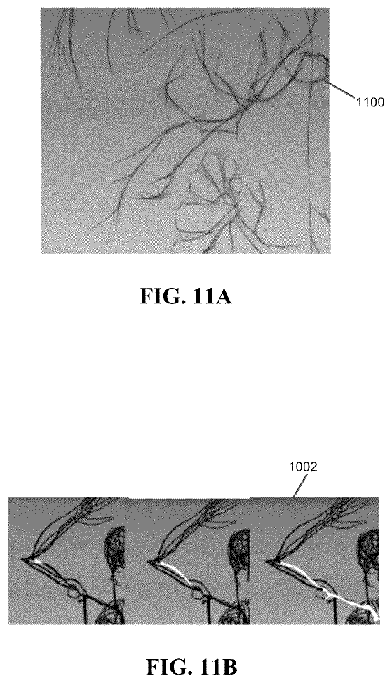

With reference to FIGS. 11A-11B and 12A-12B, shown (and further described) is the medical simulation device 103 being operative and configured to provide graphical illustration of blood flow dynamics (e.g., 1100, 1102, 1200, and 1202) in a simulated patient. In particular, and with reference to FIGS. 11A and 11B, the direction that the line is pointing determines the direction of blood flow, wherein the arteries and veins are separated and mapped such that the direction, velocity, and timing of blood flow are medically accurate based on the diameter and proximity to the heart. With particular regard to FIGS. 12A and 12B, moving blood is depicted with the blood flow patterns and prior knowledge programed inside the patient with blood moving freely throughout the body.

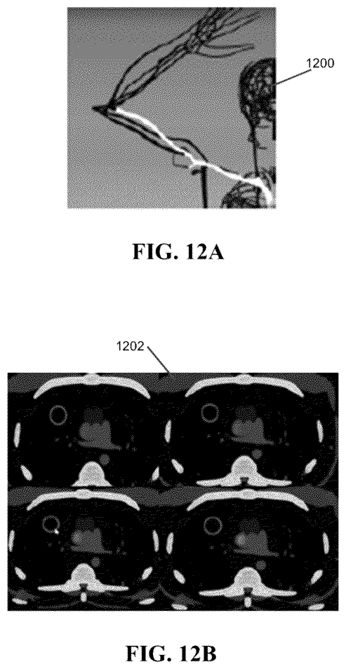

With reference now to FIG. 13, the medical simulation device 103 is shown (and further described) to provide, on a computer display (e.g., 224) translation of brightness of simulated injected dye being qualitatively converted to simulate x-ray penetration of a simulated patient 1300. For instance, the brightness of the incoming dye (0-black to 1-white range) is qualitatively converted to mimic x-ray and CT contrast between simulated body tissues. Additionally, it is to be understood and appreciated that the working cardiovascular system of the digital patient allows for the simulated injected dye to circulate through the simulated body slices for replication of actual clinical environment and workflow.

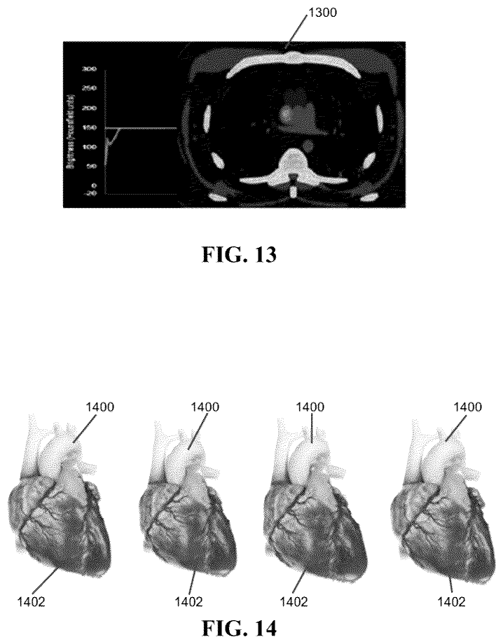

With reference to FIG. 14, the medical simulation device 103 is shown being operative and configured to provide simulated 3D modeling of the simulated patient which includes generation of a 3D model of a simulated patient's heart organ 1400 graphically illustrating differing aspects of cardiac cycle (e.g., 1402-1404) for depicting various positions of the simulated heart during contraction and relaxation (e.g., 1402-1404) enabling the simulated patient to be programmed to have certain variations in heart function, structure and/or rhythms.

Accordingly, it is to be understood and appreciated that certain embodiments of the medical simulation device/system, provides/generates, digitally simulates, various anatomical variants and physiologic processes of real humans, whereby the digital/simulated patients have individual profiles that have any number of combinations of physical or mental variables critical to acquiring successful medical imaging tests, such as (but not limited to): heart rhythm; heart function; blood flow rates and velocities; breath-hold capabilities; digestion rates; range of motion; claustrophobia; Alzheimer's disease; and tremors/restless leg syndrome.

With certain illustrated embodiments described above, it is to be appreciated that various non-limiting embodiments described herein may be used separately, combined or selectively combined for specific applications. Further, some of the various features of the above non-limiting embodiments may be used without the corresponding use of other described features. The foregoing description should therefore be considered as merely illustrative of the principles, teachings, and exemplary embodiments of the illustrated embodiments, and not in limitation thereof.

It is to be understood that the above-described arrangements are only illustrative of the application of the principles of the illustrated embodiments. Numerous modifications and alternative arrangements may be devised by those skilled in the art without departing from the scope of the illustrated embodiments, and the appended claims are intended to cover such modifications and arrangements.

What is claimed is:

1. A computer system for simulating one or more anatomical features of a simulated patient for application with a simulated medical scanning procedure, comprising:
a memory configured to store instructions;
a database repository storing a plurality of simulated patients including a plurality of anatomical features for each simulated patient for subsequent user selection,
a processor disposed in communication with the memory and database, and coupled to a computer network being configured to:
simulate a medical scanning procedure by a medical scanning device on a user selected simulated patient, wherein the user defines one or more anatomical features associated with the user selected patient; and
wherein a computer display is generated enabling user selection of a certain simulated patient, including user selection of one or more anatomical features associated with the user selected simulated patient, for medical scanning simulation with a simulated medical scanning device.

2. The computer system as recited in claim 1, wherein the processor is further configured to enable selection, for user interaction on the computer display, medical scanning settings associated with a third party vendor medical scanning device selected from a listing of certain third party vendors.

3. The computer system as recited in claim 1, wherein a virtual MRI interface is generated associated with the user selected third party vendor medical scanning device providing a same interface as associated with the actual user selected third party vendor medical scanning device.

4. The computer system as recited in claim 1, wherein the processor is further configured to simulate one or more medical modalities associated with the simulated patient, wherein the simulated medical modalities are contingent upon a patient's physiology, and wherein the simulated one or more anatomical features include three-dimensional (3D) modeling of the patient.

5. The computer system as recited in claim 4, wherein the 3D modeling includes modeling external and internal features of the simulated patient, and wherein the 3D modeling further includes independent and/or simulated patient physiology profiles, wherein the one or more physiologic features includes one or more combinations of: heart rhythm, structural variants, range of motion, cardiac output, peristalsis rate, and/or kidney functions.

6. The computer system as recited in claim 5, wherein the one or more physiologic features further includes psychologic conditions, including claustrophobia and/or nervousness.

7. The computer system as recited in claim 1, wherein the processor is further configured to to change, via user selection, an appearance and body position of the simulated patient to simulate user selected radiologic modalities, wherein the simulated user selected radiologic modalities include: positron emission tomography (PET) scans, nuclear medicine scans, and bone density scans, and, wherein the simulated patient is positionable in a plurality of different body position during a simulated medical scanning procedure.

8. The computer system as recited in claim 1, wherein the simulated medical scanning procedure includes a computer generated digital injector simulating injection of dye material into the simulated patient for application with a simulated medical scan requiring use of dye material.

9. The computer system as recited in claim 8, wherein simulating injection of dye material into the simulated patient includes simulation of dye material into the simulated patient for determining an amount of time an imaging contrast provided by the simulated dye circulates through a simulated cardiovascular system of the simulated patient.

10. The computer system as recited in claim 9, wherein the simulated digital injector is replicated on the GUI of the computer display providing one or more user interactive manipulable features for prescribing simulated injection of simulated dye material into the simulated patient, including prescribing an amount of saline and contrast fluid material to be injected into the simulated patient.

11. The computer system as recited in claim 10, wherein the simulated medical scanning procedure includes translation of brightness of simulated injected dye being qualitatively converted to simulate x-ray penetration of the simulated patient.

12. The computer system as recited in claim 1, wherein the simulated medical scanning procedure consists of a radiology scan selected from the group consisting of one or more of the following scan types: MRI; CT; Ultrasound; X-ray; Mammography; PET; Fluoroscopy; Nuclear medicine; Bone scintigraphy Mammogram; Angiography Radiography; Diagnostic imaging; Computed axial tomography′ and Virtual colonoscopy.

13. The computer system as recited in claim 1, wherein the database repository contains virtual outer shells of a plurality of simulated patients'representative of different simulated patients including: sexes; ages; body types and simulated patient bodies modelled in different positions.

14. The computer system as recited in claim 1, wherein simulating one or more anatomical features of a simulated patient includes a plurality of different human tissue types and organs, which may be segmented to improve medical accuracy of certain simulated anatomical structures of the simulated patient.

15. The computer system as recited in claim 14, wherein simulating one or more anatomical features of a simulated patient includes generation of a 3D model configured to be sliced in a multitude of directions in a 3D space for generating resulting images having an appearance to actual, non-simulated, medical images.

16. The computer system as recited in claim 1, wherein simulating one or more anatomical features of a simulated patient includes simulation of blood flow dynamics in a simulated patient.

17. The computer system as recited in claim 4, wherein the simulated 3D modeling of the simulated patient includes generation of a 3D model of a simulated patient's heart organ graphically illustrating differing aspects of cardiac cycle for depicting various positions of the simulated heart during contraction and relaxation enabling the simulated patient to be programmed to have certain variations in heart function, structure and/or rhythms.

18. The computer system as recited in claim 1, wherein the processor is further configured to implement one or more artificial learning (AI) and/or machine learning techniques for enabling the simulation of the one or more anatomical features of the simulated patient for application with the simulated medical scanning procedure.

19. A computer system for simulating one or more anatomical features of a simulated patient for application with a simulated medical scanning procedure, comprising:
a memory configured to store instructions;
a database repository storing a plurality of simulated patients including a plurality of anatomical features for each simulated patient for subsequent user selection,
a processor disposed in communication with the memory and database, and coupled to a computer network being configured to:
simulate a medical scanning procedure by a medical scanning device on a user selected simulated patient, wherein the user defines one or more anatomical features associated with the user selected patient;
simulate injection of dye material into the simulated patient for application with a simulated medical scan requiring use of dye material; and
wherein a computer display is generated enabling user selection of a certain simulated patient, including user selection of one or more anatomical features associated with the user selected simulated patient, for medical scanning simulation with a simulated medical scanning device.

20. The computer system as recited in claim 18, wherein simulating injection of dye material into the simulated patient includes simulation of dye material into the simulated patient for determining an amount of time an imaging contrast provided by the simulated dye circulates through a simulated cardiovascular system of the simulated patient, and wherein the simulated digital injector is replicated on the GUI of the computer display providing one or more user interactive manipulable features for prescribing simulated injection of simulated dye material into the simulated patient, including prescribing an amount of saline and contrast fluid material to be injected into the simulated patient.

21. A computer system for simulating injection of dye material into the simulated patient for application with a simulated medical scan requiring use of dye material for application with a simulated medical scanning procedure, comprising:
a memory configured to store instructions;
a database repository storing a plurality of simulated patients including a plurality of anatomical features for each simulated patient for subsequent user selection,
a processor disposed in communication with the memory and database, and coupled to a computer network being configured to:
simulate a medical scanning procedure by a medical scanning device on a user selected simulated patient;
simulate injection of dye material into the simulated patient for application with a simulated medical scan requiring use of dye material; and
wherein simulating injection of dye material into the simulated patient includes simulation of dye material into the simulated patient for determining an amount of time an imaging contrast provided by the simulated dye circulates through a simulated cardiovascular system of the simulated patient, and wherein the simulated digital injector is replicated on the GUI of the computer display providing one or more user interactive manipulable features for prescribing simulated injection of simulated dye material into the simulated patient, including prescribing an amount of saline and contrast fluid material to be injected into the simulated patient.
